(19) United States
(12) Patent Application Publication
(10) Pub. No.: US 2026/0364252 A1
(43) Pub. Date: Nov. 19, 2026

(54) SYSTEMS AND METHODS FOR PORTABLE AUTOMATED TRANSFUSION

(71) Applicant: ELLIOTT CALDERON, Carlsbad, CA (US)

(72) Inventor: ELLIOTT CALDERON, Carlsbad, CA (US)

(21) Appl. No.: 19/676,697

(22) Filed: May 13, 2026

Related U.S. Application Data
(60) Provisional application No. 63/805,703, filed on May 14, 2025.

(51) Int. Cl. A61M 5/148; A61M 5/172

(52) U.S. Cl. CPC A61M 5/1486; A61M 5/172; A61M 2205/07; A61M 2205/3327; A61M 2205/3331; A61M 2205/3334

(57) ABSTRACT

A portable automated infusion system includes a sleeve having a first end, a second end, a longitudinal axis, and an internal opening extending along the longitudinal axis from the first end, an outer wall having substantially low overall distensibility and extending 360° around the longitudinal axis, and configured to surround a blood bag, an inner wall having a first end and a second end, the first end of the inner wall in a sealing arrangement with a first portion of the outer wall, the inner wall having an interior portion defining the internal opening, and a substantially annular space between the outer wall and the inner wall, the system further including a pump coupled to the sleeve and configured to pump a fluid into the annular space, a pressure sensor configured to measure fluid pressure within the annular space and to output a signal related to the measured fluid pressure, and a controller configured to receive the output signal and configured to control operation of the pump.

### INCORPORATION BY REFERENCE TO ANY PRIORITY APPLICATIONS

This application claims the benefit of priority to U.S. Provisional Patent Application No. 63/805,703, filed on May 14, 2025, which is incorporated by reference herein in its entirety for all purposes. Priority is claimed pursuant to 35 U.S.C. § 119.

### BACKGROUND OF THE INVENTION

### Field of the Invention

The field of the invention generally relates to devices for bringing media into the body in a subcutaneous, intra-vascular way, including accessories therefor.

### SUMMARY OF THE INVENTION

In one embodiment of the present disclosure, a portable automated infusion system includes a blood bag including an interior containing a liquid including one or both of blood and/or a blood substitute, the blood bag further including a port configured to enable the exit of the liquid, a sleeve including a first end, a second end, a longitudinal axis, and an internal opening extending along the longitudinal axis from the first end, the internal opening configured to allow the blood bag to be close-fittingly carried therein, the sleeve including an outer wall having substantially low overall distensibility, the outer wall extending 360° around the longitudinal axis and configured to surround the blood bag, when the blood bag is placed within the internal opening of the sleeve, an inner wall having a first end and a second end, the first end of the inner wall in a sealing arrangement with a first portion of the outer wall at or adjacent the first end sleeve and the second end of the inner wall in a sealing arrangement with a second portion of the outer wall spaced from the first portion in a direction toward the second end of the sleeve, the inner wall having an interior portion defining the internal opening, and a substantially annular space between the outer wall and the inner wall, the system further including a pump coupled to the sleeve and configured to pump a fluid into the annular space, a pressure sensor configured to measure fluid pressure within the annular space and to output a signal related to the measured fluid pressure, and a controller configured to receive the output signal and configured to control operation of the pump.

In another embodiment of the present disclosure, a portable automated infusion system includes a fluid bag including an interior containing a liquid, the fluid bag further including a port configured to enable the exit of the liquid, a sleeve including a first end, a second end, a longitudinal axis, and an internal opening extending along the longitudinal axis from the first end, the internal opening configured to allow the fluid bag to be close-fittingly carried therein, the sleeve including an outer wall having substantially low overall distensibility, the outer wall extending 360° around the longitudinal axis and configured to surround the fluid bag, when the fluid bag is placed within the internal opening of the sleeve, an inner wall having a first end and a second end, the first end of the inner wall in a sealing arrangement with a first portion of the outer wall at or adjacent the first end sleeve and the second end of the inner wall in a sealing arrangement with a second portion of the outer wall spaced from the first portion in a direction toward the second end of the sleeve, the inner wall having an interior portion defining the internal opening, and a substantially annular space between the outer wall and the inner wall, the system further including a pump coupled to the sleeve and configured to pump a fluid into the annular space, a pressure sensor configured to measure fluid pressure within the annular space and to output a signal related to the measured fluid pressure, and a controller configured to receive the output signal and configured to control operation of the pump.

In yet another embodiment of the present disclosure, a portable automated infusion system includes a sleeve including a first end, a second end, a longitudinal axis, and an internal opening extending along the longitudinal axis from the first end, the internal opening configured to allow a blood bag to be close-fittingly carried therein, the sleeve including an outer wall having substantially low overall distensibility, the outer wall extending 360° around the longitudinal axis and configured to surround the blood bag, when the blood bag is placed within the internal opening of the sleeve, an inner wall having a first end and a second end, the first end of the inner wall in a sealing arrangement with a first portion of the outer wall at or adjacent the first end sleeve and the second end of the inner wall in a sealing arrangement with a second portion of the outer wall spaced from the first portion in a direction toward the second end of the sleeve, the inner wall having an interior portion defining the internal opening, and a substantially annular space between the outer wall and the inner wall, the system further including a pump coupled to the sleeve and configured to pump a fluid into the annular space, a pressure sensor configured to measure fluid pressure within the annular space and to output a signal related to the measured fluid pressure, and a controller configured to receive the output signal and configured to control operation of the pump.

In still another embodiment of the present disclosure, a method of transfusing a patient in a combat setting includes providing a portable automated infusion system as disclosed herein, accessing a blood vessel of the patient with a blood line, coupling the interior of the blood bag to the blood line, and allowing the pump to deliver at least some of the blood into the patient at an injection patent that is controlled by the controller.

### BRIEF DESCRIPTION OF THE DRAWINGS

FIG. 1A is a perspective view of a portable automated infusion system, according to an embodiment of the present disclosure.

FIG. 1B is an exploded view of the portable automated infusion system of FIG. 1A.

FIG. 2A is a cross-sectional view of the portable automated infusion system taken along line 2 of FIG. 1A in a first extent of inflation, according to an embodiment of the present disclosure.

FIG. 2B is a cross-sectional view of the portable automated infusion system taken along line 2 of FIG. 1A in a second extent of inflation, according to an embodiment of the present disclosure.

FIG. 2C is a cross-sectional view of the portable automated infusion system taken along line 2 of FIG. 1A in a third extent of inflation, according to an embodiment of the present disclosure.

FIG. 3 is a perspective view of the portable automated infusion system, according to an embodiment of the present disclosure.

FIG. 4 is a front view of the portable automated infusion system of FIG. 3.

FIG. 5 is a sectional view of the portable automated infusion system of FIG. 3.

FIG. 6 is a top view of the portable automated infusion system of FIG. 3.

FIG. 7A is a perspective view of a portable automated infusion system, according to an embodiment of the present disclosure.

FIG. 7B is an exploded view of the portable automated infusion system of FIG. 7A.

FIG. 8A is a front view of the portable automated infusion system of FIG. 7A.

FIG. 8B is a cross-sectional view of the portable automated infusion system of FIG. 8A, taken along line 8B.

FIG. 8C is a sectional view of the portable automated infusion system of FIG. 8A, taken along line 8C.

FIG. 8D is a perspective view of the portable automated infusion system of FIG. 8A.

FIG. 9A is a front view of the portable automated infusion system, of FIG. 8A.

FIG. 9B is top view of the portable automated infusion system of FIG. 8A.

FIG. 9C is a side view of the portable automated infusion system of FIG. 8A.

FIG. 9D is a perspective view of the portable automated infusion system of FIG. 8A.

FIG. 10A is a perspective view of a portable automated infusion system, according to an embodiment of the present disclosure.

FIG. 10B is an exploded view of the portable automated infusion system of FIG. 10A.

FIG. 11A is a front view of a portable automated infusion system of FIG. 10A.

FIG. 11B is a cross-sectional view of the portable automated infusion system of FIG. 11A, taken along line 11B.

FIG. 11C is a sectional view of the portable automated infusion system of FIG. 11A, taken along line 11C.

FIG. 11D is a partially cut-away perspective view of the portable automated infusion system of FIG. 11A.

FIG. 12 is a flow chart of a feedback loop of a portable automated infusion system, according to an embodiment of the present disclosure.

### DETAILED DESCRIPTION OF THE ILLUSTRATED EMBODIMENTS

The present disclosure generally relates to devices for bringing media into the body in a subcutaneous, intra-vascular way, including accessories therefor. The devices include embodiments for portable infusion or transfusions systems that are automated. The applications include use in combat or battle situations for traumatic injuries. The applications include use in emergency operations, including paramedic.

Trauma to humans or animals can happen in locations that are remote from any hospital or medical facility. Oftentimes, traumatic injuries can cause significant blood loss, which can be life threatening. Some examples include trauma from mountain climbing, ice climbing, base jumping, skiing, hunting, or any sport, recreational activity, or transportation that takes place in rural or secluded areas. Wartime conflicts, or even military training with live ammunition can result in trauma in locations that are either remote and/or cannot be accessed by emergency personnel. In combat situations, even an urban setting can become a “no man's land,” with standard medical facilities empty or closed, due to the inherent danger of the conflict.

In these situations, traumatic injuries that require blood transfusions, including whole blood, red blood cells, platelets, or plasma, can be difficult to administer. This also can include blood substitutes or even fluids for volume injection, such as Ringer's solution, normal saline, or an infusion liquid comprising a medicant. If active shooting, strafing, or bombing is occurring in the area, and/or if the conflict is in an area containing landmines, the delivery of care to an injured military or non-military person or other animal can be made difficult or at times nearly impossible. A combat medic can sometimes have the ability to quickly access a vein, for example by insertion of a hypodermic needle into the vein, but may not have the time or safety to stay with the patient as a desired volume of blood or other fluid is injected into the patient. Some similar challenges occur in non-combat situations in rural areas, including mountainous, desert, island, or jungle areas, that do not have local medical facilities. A portable automated transfusion/infusion system is described herein to aid the therapeutic blood/fluid infusion in these types of patients in these types of situations and locations.

### Device

### Sleeve

The device can be characterized as an inflatable sleeve of a high-tensile material. The sleeve is made up of two layers, an exterior layer and an interior layer with space in between to allow for air to flow in between the two layers. The two layers of the sleeve are sealed together at the ends to not allow any air to escape. The air that flows in between the exterior and interior layer can only come in and out through designated inflation and deflation portions. The two layers are composed of high-tensile materials. The interior layer is configured to be inflated in order to compress a blood bag or other fluid bag to pressurize the bag's contents and aid infusion/perfusion of the contents into the vasculature of a patient.

### On/off Switch

The device has a switch that can turn the device on or off. The location of the switch is found in a designated area on the device that can be reached by the user. The switch can also be a knob or any form that can turn the device on. The switch can also be part of another button already on the device and does not have to be separate. In some embodiments, the on/off switch is configured to turn the power supply to the electronics on and off. In some embodiments, the on/off switch is configured to place the device in a state that is ready to receive one or more instructions. Thus, for example, the sleeve can then be inflated.

### Battery

A Battery is used to provide power to the electronic components of the device. The battery is meant to be found inside the housing but can also be located outside the housing. The battery can be rechargeable or non-rechargeable. The battery is able to provide power to all components necessary in the configuration of the device. The battery can be small enough to fit within the sleeve without interfering with the inflation of the inner layer.

### Button, Start/Input

The device has an input button, switch, knob, or toggle that causes the device to start the inflation process. The button sends a signal to a microcontroller or logic gate, logic gates, or series of logic gates that start the inflation process. The button does not need to be a separate entity but can be part of other buttons, switches knobs, or toggle inputs into the system. The function of this is to start the inflation process.

Toggle, Knob Potentiometer, or Switch Control Input

The device has a toggle button, knob potentiometer, switch control input, or toggle buttons that allow the user to interact with a GUI (graphic user interface) of the device. The button takes the input of the user and allows the user to navigate any menu in the GUI as well as the ability to set a target pressure and/or volume and/or rise time that the device will inflate to. The menus that can be navigated are loaded onto the device. If no menus are present the user can still use the toggle to set a target pressure and/or volume and/or rise time for the device that prompts the device to begin to inflate until that target is reached.

### Component Housing

The component housing is configured to hold most or all the components necessary to run the device. The components include, but are not limited to, a microcontroller, sensors, a battery, any button that is used for a button-specific function, a transmitter or receiver, and a screen/display/GUI. The component housing can be located in between the exterior and interior sleeve layers of the device but in other embodiments can be located at the exterior of the device. The position of the component housing will be where it is best suited for the user to access the components and interact with the GUI The component housing is an enclosure that can include all components inside of it or can allow for a way to interact with any other components that are not inside the component housing. The component housing although found in between the two layers will be configured in a shape that creates a seal so that the sleeve can inflate without any leaks.

### Microcontroller

The device does not require a microcontroller, but in the configurations where there is a microcontroller the purpose of the microcontroller is to control all inputs and outputs for the device and to handle any logic or algorithm needed to operate the device. The microcontroller has the necessary functions needed to run a GUI on the device. In some embodiments, a separate display microcontroller is utilized.

### Sensor

The sensor or sensors on the device are configured to detect the pressure changes inside of the bag (e.g., between interior and exterior layers). The sensor can comprise a pressure transducer/gauge or a strain gauge. The role is to translate the interaction between the expansion of the bag and the pressure applied on the blood bag so that the user can know the flow rate at which the fluid within the blood bag is being forced/pushed out. The sensors can be located in between the exterior layer and the interior layer of the sleeve or can be found on the exterior layer.

### Transmitter and Receiver (or Transceiver)

The device can comprise a transmitter or a receiver, or in some embodiments a transceiver, in order to communicate data via a communication protocol of the user's choice. Some of the data that will be passed includes but is not limited to the flow rate at which the fluid is coming out of the blood bag, time until the procedure is finished or is to be finished, battery life status, or particular malfunctions that could be occurring with the device. The data transmitted can be used for any purpose that the user desires. The transmitter will be found inside the component housing or can be located outside of the exterior layer for better transmission of information.

### Reinforcement Bands

In some embodiments, one or more reinforcement bands are utilized. The purpose of the reinforcement bands is to add extra tensile strength to the exterior layer of the so that as the space between the interior layer and exterior layer is pressurized, the air that would otherwise distend the exterior layer now substantially distends the interior layer only. Thus, the bag it is compressed at a quicker rate, reaching the desired pressure quicker. The reinforcement bands are made of an elastomeric material having a relatively high modulus, or of a non-elastomeric material, and are found lining the exterior layer but in between the exterior layer and the interior layer. In other embodiments, the bands substantially surround the exterior layer.

### Screen (Visual)

The screen is meant to show the user of the device the GUI. The screen is meant to show the menu loaded onto the device, but also it is meant to show the flow rate at which the fluid is flowing. On the screen, the user can see the target flow rate they can set before starting the device. The screen can be located within or on the component housing, and/or on the exterior layer of the sleeve facing outward. In some embodiments, the screen can be a touch-sensitive screen and in some embodiments can be substantially stiff. In other embodiments, the screen is flexible. The screen will communicate with the microcontroller in a device having a microcontroller. A screen is also not required for the device. Any configuration of the device without the screen will have another way of communicating to the user the flow rate at which the blood is flowing.

### Fluid Bag Holder

The fluid bag is held inside the sleeve before the sleeve applying pressure via a hook that is placed on the outside of the interior layer, facing into the through gap, where the fluid bag slides in. The hook is meant to hold the blood bag in whatever position the sleeve is placed. Once hooked inside, the fluid bag will not slip out. Another method of holding the bag within the space can be through the use of an array of slip-resistant dots or other-shaped elements that are found on the outside of the interior layer, facing into the gap where the fluid bag comes in. They are configured to contact the exterior of the fluid bag, to grip or otherwise add frictional forces to the bag

### Air Release Valve

The device can in some embodiments have an air release valve. In the configurations that it does, the air release valve will be located on the exterior layer of the bag with access to the middle of the sleeve between the exterior layer and the interior layer. The purpose of the valve is to release the air. The other way that the air can be released is via the motor/air pump. The motor moves air given that its nozzle into the sleeve is positioned in between the exterior and interior layer of the sleeve.

### General Functionality of the Device

The device is turned on and the user is prompted to insert the fluid bag. The fluid bag is slid into the sleeve and is held by the hook, the dots, and/or any other way to hold the bag. The user then toggles through the flow rate they would like to set for the transfusion. Once the flow rate is chosen the user will press the button that will start the inflation process. The motor will start and the sleeve will inflate, applying pressure to the fluid bag and dispensing the liquid found inside the bag. The sensor will measure the change of inflation of the sleeve or the pressure of the air inside the bag. The sensor will output a signal to relay that information back to the microcontroller which will then determine whether the target pressure on the bag has been reached for the desired flow rate. Once the target pressure is reached, a setpoint will be identified by the microcontroller, and the motor will be turned off by the microcontroller. As the sleeve loses pressure due to the loss of fluid, the microcontroller will instruct the motor to turn back on until the desired pressure is reached. Once the microcontroller detects that all the fluid has been pushed out, the device alerts the user via the transmission of data or by displaying the information on the screen. The device will then stop and reset. Alternatively, an alarm is triggered, comprising a sound, a visual aid (e.g., light, LED), or a mechanical movement such as a vibration. The air that is inflated can either be released via the motor pumping out the air or via a nozzle that releases air.

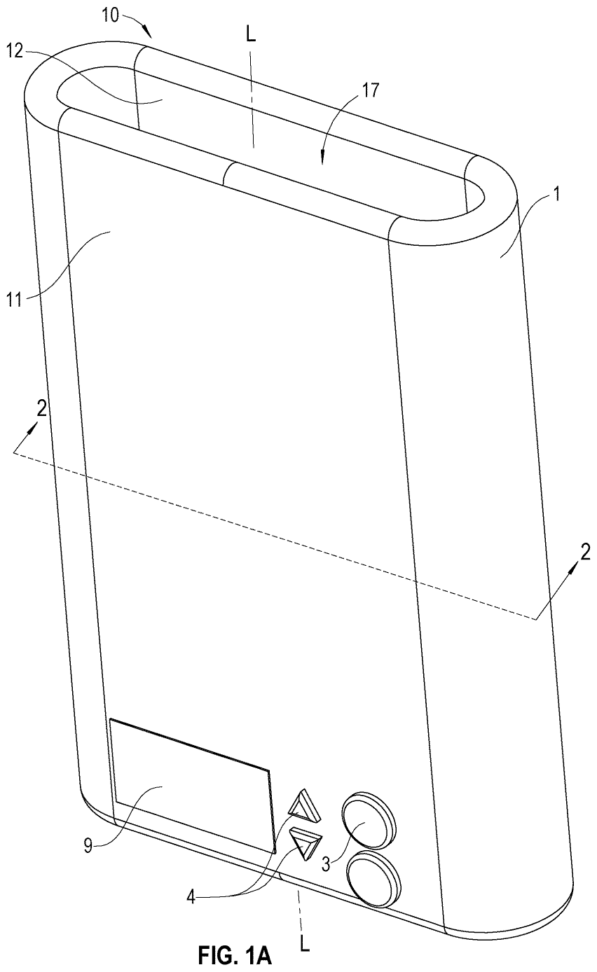

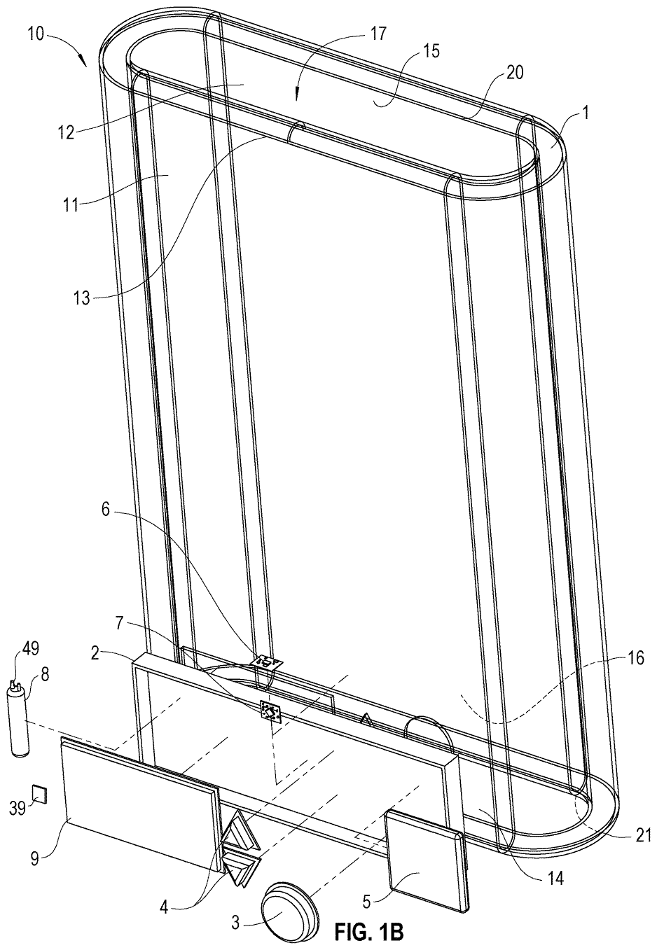

FIGS. 1A-1B illustrate a portable automated infusion system 10 comprising a sleeve 1, and a component housing 2 which carries components of the system 10. The sleeve 1 comprises an outer wall 11 and an inner wall 12. Each of the outer wall 11 and the inner wall 12 extends 360° around the sleeve 1, in relation to sleeve longitudinal axis L. In oval cross-sectional shape is illustrated in the embodiment of FIGS. 1A-1B, but in alternative embodiments, can comprise a rectangular shape, or an elliptical shape, or even a substantially circular shape. The outer wall 11 comprises a first end 13 and a second end 14. The inner wall 12 comprises a first end 15 and a second end 16. An internal opening 17 extends between the first end 15 of the inner wall 12 and the second end 16 of the inner wall 12, through the sleeve 1. The internal opening 17 in some embodiments passes completely through the sleeve 1. In other embodiments, the internal opening 17 extends between one end of the sleeve 1 but not through the other end of the sleeve 1. For example, through the bottom end but not the top end. Either way, the internal opening 17 is configured for the placement of a blood bag 19 (or other fluid bag) containing blood 18 (or another fluid, e.g., liquid or in some cases, even a gaseous component or mixture) (see FIGS. 2A-2C). The inner wall 12 is sealed to the outer wall 11 at a top seal portion 20. The inner wall 12 is also sealed to the outer wall 11 at bottom seal portion 21. Each of the seal portions 20, 21 extend 360° around the longitudinal axis L. In the particular embodiment of FIGS. 1A-2C, the 360° seal portions 20, 21 form an oval shape transverse to the longitudinal axis L. From the top seal portion 20 to the bottom seal portion 21, an annular space 22 extends longitudinally between the outer wall 11 and the inner wall 12.

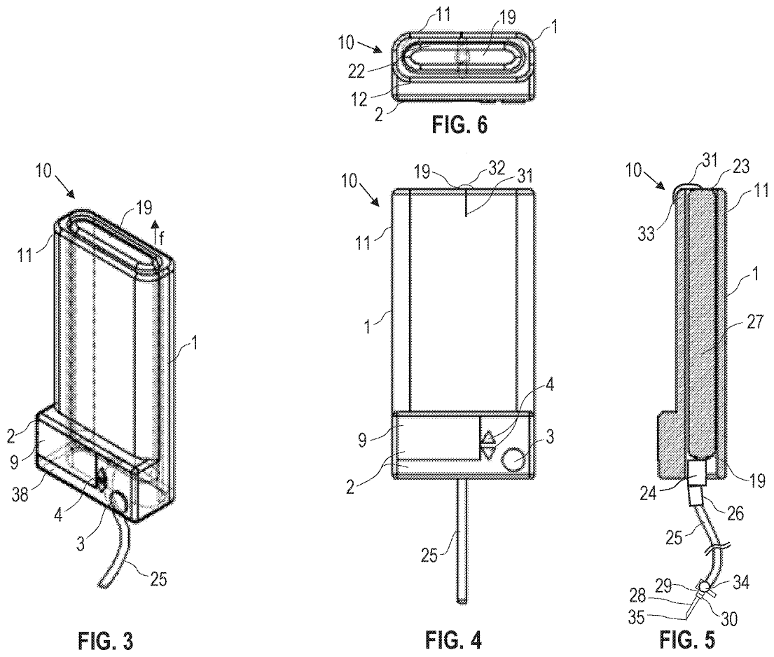

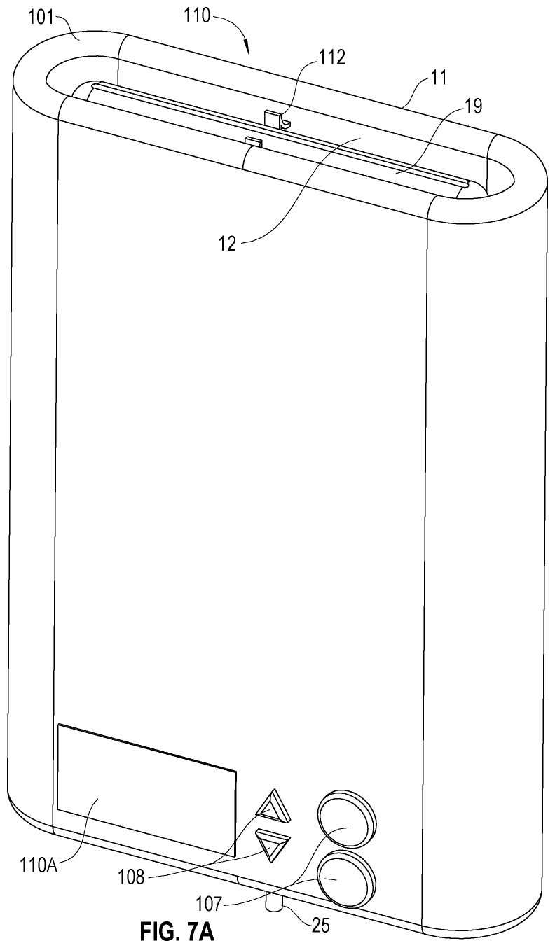

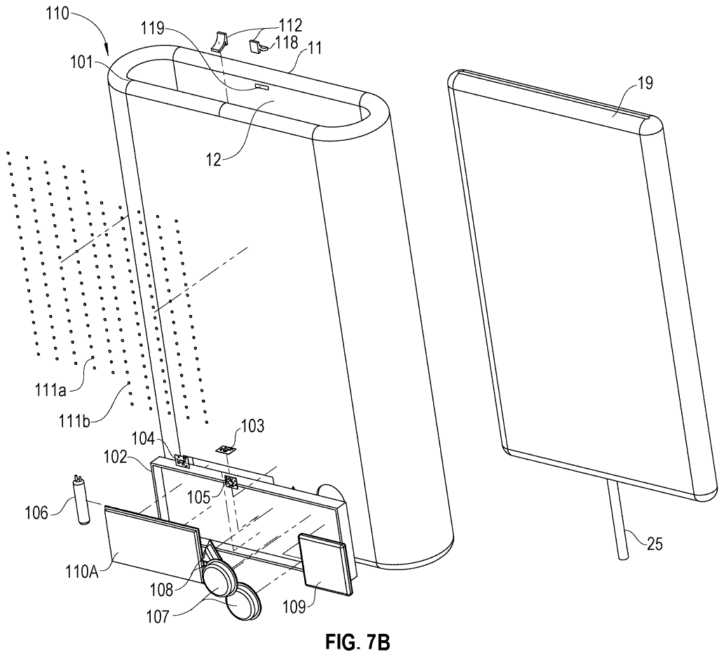

The annular space 22 is configured for air or other gaseous (or non-gaseous) fluids to be injected therein. The fluids can be compressible or substantially incompressible. By increasing the pressure of a fluid within the annular space 22, the blood bag 19 can be controllably compressed via externally applied pressure from the inner wall 12. The blood bag 19 comprises a bag portion 23, and (turning to FIG. 5) a port 24. The port 24 is configured to controllably access the interior 27 of the blood bag 19. This is achieved via an infusion line 25 having a spike 26. The infusion line 25 can be coupled to a hypodermic needle 28 (e.g., via luer connectors 29, 30). The hypodermic needle 28 has a sharp needle tip 35 configured for puncturing the skin and a blood vessel wall (e.g., vein) of a patient, to insert the needle therein. The luer connector 29 can include or can be coupled to a valve 34, which can comprise a one-way stopcock, a two-way stopcock, or a three-way stopcock, or another type of valve that can open and close to start and stop the flow of blood out of the blood bag 19. Though the word “annular” is used in the term “annular space 22,” it does not need to be circular. In some embodiments, it can be circular, but in the embodiment of FIGS. 1A-6, it is more oval in the uninflated state, and then varies as the blood bag 19 is put in place, and as the inflation progresses. The term “annular” is used here to describe something extending peripherally around an inner element (i.e., inner wall 12) and extending around the interior of the outer wall 11.

In some embodiments, an alternate hook 31 is coupled to an upper portion 32 of the blood bag 19. The hook 31 is curved, or U-shaped, or J-shaped, of V-shaped and can comprise a wire form, or a molded, cast, or otherwise formed element. The hook 31 is configured to hold the blood bag 19 vertically in place (static) by hooking onto a top edge 33 of the sleeve 1. Thus, the hook 31 provides an equal and opposite force to the force of gravity, and whatever downward force may or may not be added via the inflation of the inner wall 12 and the compression of the blood bag 19.

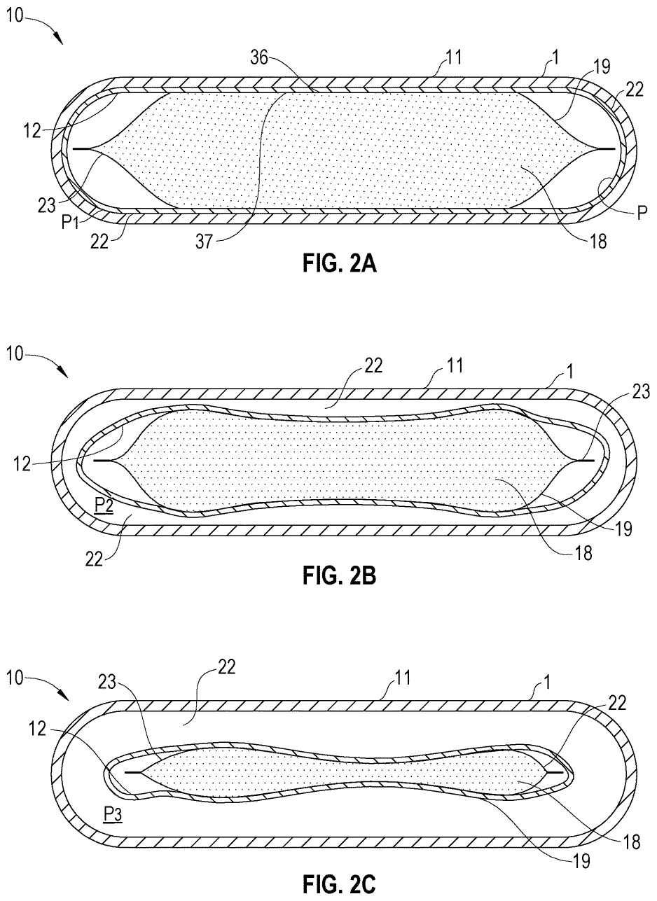

The internal opening 17 can be configured such that in a generally non-inflated state (see FIG. 2A), the blood bag 19 is held closely, or slightly snugly, or very snugly. For example, in the non-inflated state (FIG. 2A), ignoring the weight of the blood bag 19, an additional longitudinally-oriented frictional force f (FIG. 3) between an outer surface 36 of the blood bag 19 and an inner surface 37 of the inner wall 12 is between about 20 grams and about 600 grams, or between about 50 grams and about 500 grams, or between about 100 grams and about 400 grams. In certain situations, there might not be enough stability between the patient and the surroundings (hillside, berm, foxhole) to rely completely on the weight of the blood bag 19 alone. Thus, the inherent friction added by the inner surface 37 of the inner wall 12, as well as the amount of fit designed into the size of the inner wall inner dimensions (e.g. perimeter length p), can be optimized in manufacture to provide the desired frictional force f. The internal opening 17 is thus defined by the dimensions of the inner wall 12. Materials with a tacky or slightly tacky surface can be utilized to add more frictional force f, such as ethylene vinyl acetate, polyvinyl chloride, polyether block amide, polyurethane, or low-density polyethylene.

A pump 8 (FIG. 1B) comprises an air pump having a motor to drive an impeller or other mechanisms. The pump is coupled to a valve 38 (FIG. 3) configured to allow external air (e.g., ambient air) to be pulled into the annular space 22 by the pump 8. In some embodiments, the pump comprises 12V Vacuum Pump (Hyudo 12V 1.8a 30 W). A pressure sensor 6 is carried within the annular space 22, and can be held statically on/in the component housing 2. The pressure sensor 6 is oriented such that it is capable of measuring the fluid pressure (e.g., gas pressure) inside the annular space 22. The pressure sensor 6 is coupled to a controller, such as a microcontroller 7, which can comprise a Nano V3.0 (Lafvin 5V 16M micro controller board). The microcontroller 7 can include a microprocessor and is configured or configurable or programmable to receive a signal output from the pressure sensor 6 indicative of pressure, and to make calculations and control other elements utilizing the signal or the processed signal. The microcontroller 7 is configured to start and stop the pump 8, and the increase and decrease the speed of the motor of the pump 8. The microprocessor 7, pressure sensor 6, and pump 8 together form a feedback loop. In some embodiments, the microcontroller 7 is configured to receive a mechanical signal. This may include, for example, movement of fluid representing a pressure differential, such as that associated with a manometer that is coupled to the microcontroller, or via a movable rod that is coupled to a mechanical valve or a solenoid.

A memory 39 can be utilized to store information, such as a maximum allowable pressure within the annular space 22, or a target mean pressure within the annular space 22. The memory 39, in some embodiments, can store one or more preset or user-compiled or user-set routines, such as a particular pattern for pump speed (e.g., motor speed) over time, or for target fluid pressure in the annular space 22, or for target fluid flow rate or fluid flow velocity. A battery 5 is carried with the other components in the component housing 2, and is configured to power the other electronic components, as necessary. In some embodiments, the battery 5 is rechargeable. In some embodiments, the battery is permanent. In some embodiments, the battery 5 is replaceable. A button 3 can comprise an on button, or an on/off button, or an off button. A pair of toggle buttons 4 are used to increase or decrease one or more parameters, for example, target internal pressure of the annular space 22, maximum allowable pressure of the annular space 22, target flow rate injected by the pump 8, pump 8 motor speed, input voltage supplied to the pump 8, fluid flow rise time, or fluid pressure rise time. The microcontroller 7 can also be configured to open and close the valve 38 or another valve, for example, to allow pressurized air within the annular space 22 to exit the annular space 22, to thus lower the internal pressure. In other embodiments, the microcontroller 7 can include one or more setting, for example embedded and stored in non-volatile memory, that includes one or more routines. These one or more routines can comprise a particular pattern for pump speed (e.g., motor speed) over time, or for target fluid pressure in the annular space 22, or for target fluid flow rate or fluid flow velocity.

In some embodiments, there may be an additional flow sensor 49, or the flow sensor 49 can be used instead of the pressure sensor 6. For example, the flow sensor can be a Doppler sensor that is capable of determining the fluid entry velocity exiting the pump 8. The microcontroller 7 would, in that particular embodiment, also be capable of receiving a signal from the flow sensor 49, and operating the pump 8 and the flow sensor 49 in the feedback loop.

A screen 9 is carried by the component housing 2, and is configured to provide visual information to a user. This may include status (“pump running,” “pump off,” “pump malfunction,” “exceeding maximum pressure”) and data (“120 psi,” [air pressure in annular space 22] “250 ml/minute,” [blood transfusion rate] “5 minutes, 35 seconds” [total time transfused]). In some embodiments, the screen 9 provides a graphical user interface (GUI), that can also include touch sensitivity, such as capacitive touch sensitivity or resistive touch sensitivity. The GUI can also incorporate a graphical version of the controls, such as the buttons 3, 4. In some embodiments, Bluetooth® technology (Bluetooth Special Interest Group, Kirkland, WA, USA) can be utilized. In some embodiments, the GUI is voice-activated, thus allowing the user to deliver voice commands. In some embodiments, the GUI is configured to couple to a network of a plurality of devices.

In some embodiments, the microcontroller 7 allows the portable automated infusion system 10 to operate autonomously. In some embodiments, an additional artificial intelligence (AI) chip (not shown) is coupled to the microcontroller 7, and is configured to modify presets, based on internal information (experience of use), or from external information.

The outer wall 11 can in some embodiments comprise a substantially low-distensibility polymer, such as polyester, polyamide, polyethylene terephthalate (PET), and can have an outer wall thickness of between about 0.025 mm and about 30 mm, or between about 0.025 mm and about 20 mm, or between about 1 mm and about 10 mm. In some embodiments, the outer wall 11 can have a generally elliptical shape, or a generally oval shape, or a generally rectangular shape, or a generally circular shape, in relation to a cross-section of the longitudinal axis L. In some embodiments, the outer wall comprises a front portion and a back portion that can be statically coupled together. They can be permanently coupled together via thermal bonding (e.g., heat sealing), hot melt, adhesive bonding, epoxy bonding, welding, brazing, soldering, or connection via fasteners. The fasteners can include rivets, bolts, screws, anchors, nails, clips, or clamps. In some embodiments, the outer wall 11 can be molded, for example injection molded, blow molded, insert molded, compression molded, or rotational molded. In some embodiments, the outer wall 11 can be thermoformed, or can be produced by additive manufacturing, such as: VAT photo polymerization, material jetting, powder bed fusion, material extrusion, binder jetting, sheet lamination, or directed energy deposition. In some embodiments, the outer wall 11 can be manufactured by polymer extrusion. The outer wall 11 can be chosen to be substantially rigid.

Turning to FIG. 2B, the increase in the internal pressure in the annular space 22 is shown in the intermediate stage, and an hourglass shape begins to occur in the inwardly distending inner wall 12 as it is acted upon by the increasing pressure. This hourglass shape does not always occur, and depends on the size, shape, and thickness of the blood bag 19 as well as the amount of blood remaining within the blood bag 19 and the temperature of the blood bag 19 and its contents. FIG. 2C illustrates a further increase in the pressure within the annular space 22 and a latter state wherein the blood bag 19 has a smaller amount of blood left therein. In some embodiments, an additional hand pump (not shown) can be added to allow manual control of the internal pressure of the annular space 22. At pressure P1=0 (FIG. 2A), the inner wall 12 is not substantially pushed inwardly from the outer wall 11. At positive pressure P2 (FIG. 2B), enough air has been forced inside to create a sizeable volume of the annulus 22 and force blood from the blood bag 19. At further increased pressure P3 (FIG. 2C), the volume of the annulus 22 has been further increased. In FIG. 2B less than one-half of the blood has been expressed from the blood bag 19. In FIG. 2C, more than one half of the blood has been expressed from the blood bag 19.

The inner wall 12 in some embodiments comprises a low distensibility polymer, or a medium distensibility polymer, such as low-density polyethylene (LDPE), medium-density polyethylene (MDPE), other polyolefins, polyamides, polyether block amide, polyvinyl chloride, or polyurethane. In some embodiments, the inner wall 12 comprises a composite structure, such as a polymer wall combined with a braided/woven mesh. In some embodiments, the mesh comprises a polyamide mesh or a polypropylene mesh.

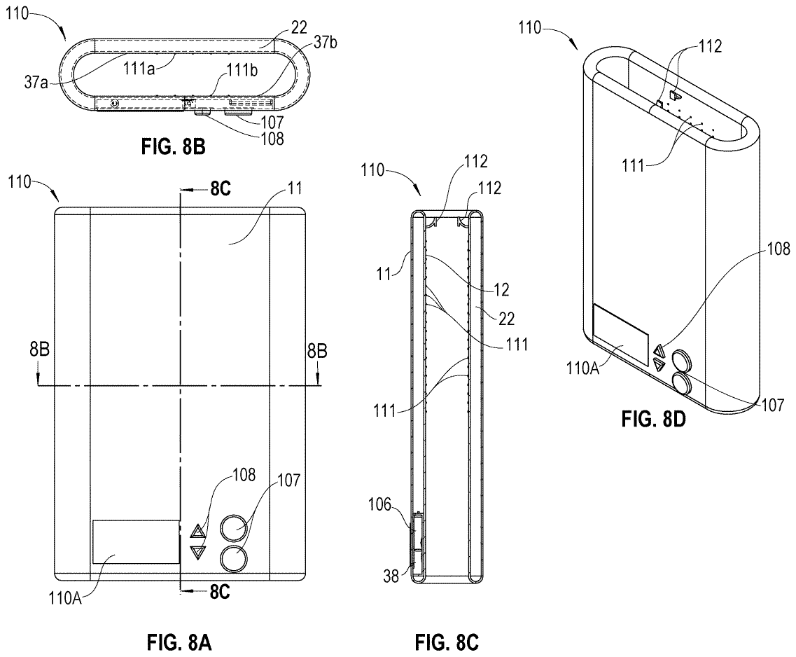

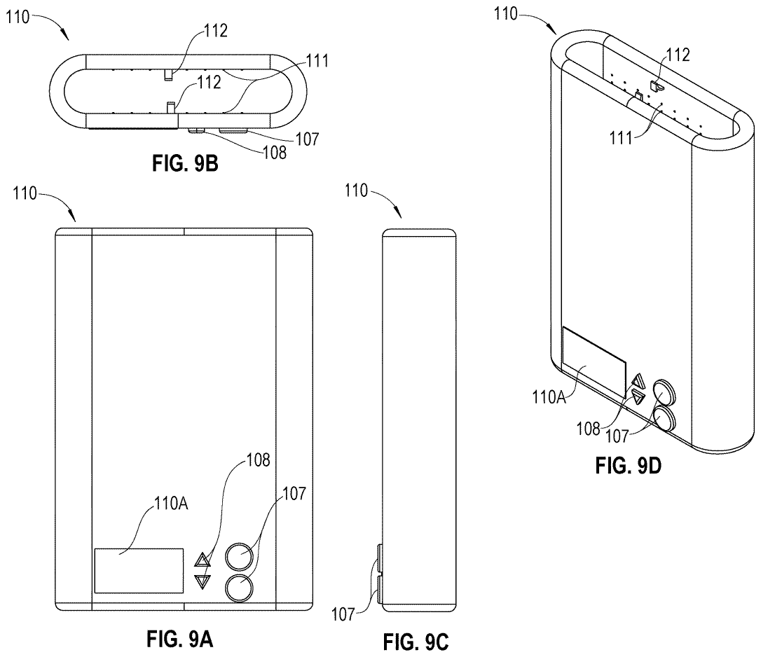

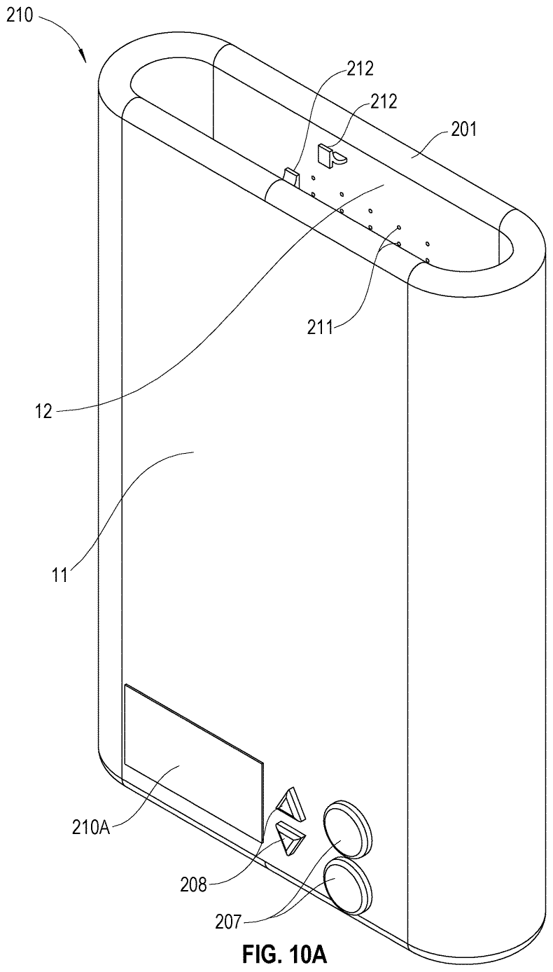

FIGS. 7A-9D illustrate another embodiment of a portable automated infusion system 110, also comprising a sleeve 101, and a component housing 102 which carries components of the system 110. The components include a pressure sensor 103 (similar to the pressure sensor 6), a microcontroller 104 (similar to the microcontroller 7), an air pump 106 (similar to the air pump 8), a battery 109 (similar to the battery 5), toggle buttons 108 (similar to the toggle buttons 4), and a screen 110A (similar to the screen 9). The on/off button 3 has been replaced by two buttons 107. They can be configured as: one for ON and the other for OFF. The outer wall 11 and inner wall 12 are configured and function similarly to those in the portable automated infusion system 10, as do the other components.

A transceiver (or transmitter-receiver duo) 105 is configured to send and receive data and control information, and to communicate with the microcontroller 104. As an adjunctive element, or to replace the specific frictional functionality of the inner surface 37 of the inner wall 12 with the outer surface 36 of the blood bag 19, a plurality of slip resistant elements 111 are carried on the inner surface 37 of the inner wall 12. Slip resistant elements 111a are adhered to first side inner wall surface 37a, and slip resistant elements 111b are adhered to first side inner wall surface 37b. The slip resistive elements 111 can comprise low-lubricity dots. In some embodiments, the slip resistive elements 111 have a convex side and a flat side. The flat side can comprise an adhesive strip in order to attach the slip resistive elements 111 to on the inner surface 37 of the inner wall 12. The plurality of slip resistive elements 111 work together to hold the blood bag 19 in place via friction and/or micro engagement with the outer surface 36 of the blood bag 19. In some cases, the micro engagement is proportional to the total amount that the blood bag 19 is filled with blood, thus engaging more when the blood bag 19 is heavier (containing a larger mass of blood). The friction is increased with increased engagement (element 111 deforming the wall of the blood bag 19 more), because the normal force of engagement is increased (with a more full blood bag 19).

A hook or holder 112 is carried on the sleeve 101, one on each side. The holder 112 comprises a hooked shape, and can be adhesively attached, snapped in place, or attached with fasteners. The holder 112 is configured to hook through an aperture 119 at the top of a blood bag 19 in order to hold it in place. A tongue 118 engages within the aperture 119 to be bonded and/or to be snapped in place.

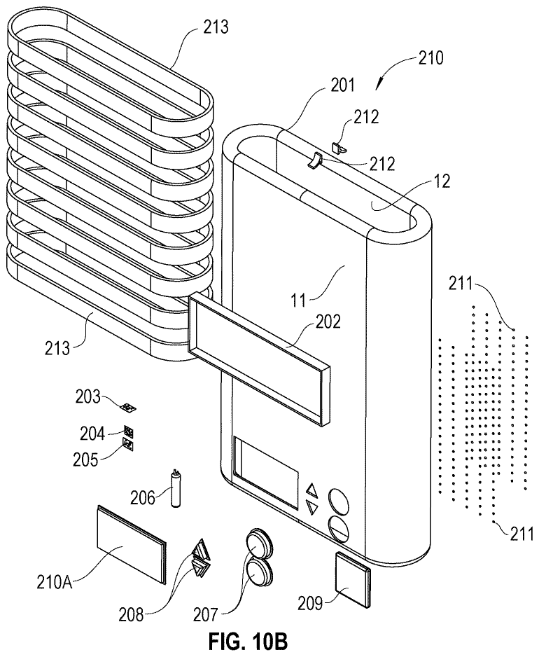

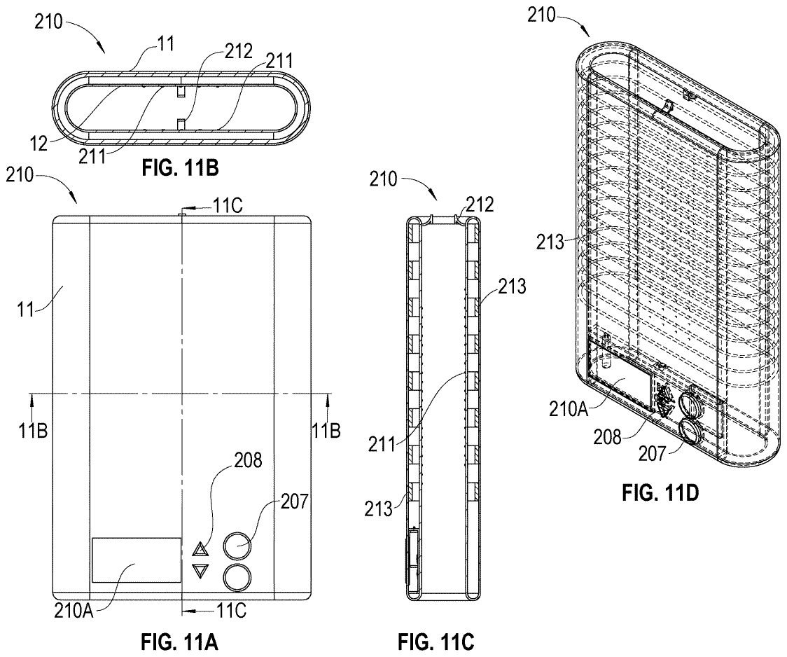

FIGS. 10A-11D illustrate another embodiment of a portable automated infusion system 210, also comprising a sleeve 201, and a component housing 202 which carries components of the system 210. The components include a pressure sensor 203 (similar to the pressure sensor 6), a microcontroller 204 (similar to the microcontroller 7), a transceiver 205 (similar to the transceiver 105), an air pump 206 (similar to the air pump 8), two buttons 207 (similar to the two buttons 107), a battery 209 (similar to the battery 5), toggle buttons 208 (similar to the toggle buttons 4), and a screen 210A (similar to the screen 9). The outer wall 11 and inner wall 12 are configured and function similarly to those in the portable automated infusion system 10, as do the other components.

There are also slip resistant dots 211 and a bag holder 212 which function similar to those of the portable automated infusion system 110. The outer wall 11 can comprise a rigid material, but can also comprise a less rigid material, because there are one or more reinforcement bands 213 which are embedded within the outer wall 11 in order to maintain its overall distensibility low. In the embodiment shown in FIGS. 10A-11D, there are eight reinforcement bands 213. In some embodiments, the reinforcement bands 213 can be evenly separated from each other. In other embodiments, the reinforcement bands 213 can be unevenly separated. For example, they can be closer to each other at a lower portion of the outer wall 11, and further apart at an upper portion. In alternative embodiments, the one or more reinforcement bands 213 can be wrapped around the outside of the outer wall 11. The one or more reinforcement bands 213 also serve to add overall strength to the outer wall 11, thus to maintain higher pressures, and maintain them for extended periods of time. The one or more reinforcement bands 213 also make the portable automated infusion system 210 for use in trying situations (e.g., combat or rescues/emergencies) and environmental elements. The one or more reinforcement bands 213 can comprise a high tensile material, such as stainless steel, steel, a high strength polymer such as polyester, polyamide, polyether ether ketone (PEEK). In some embodiments, there are one to twenty reinforcement bands 213, or two to ten reinforcement bands 213, or three to eight reinforcement bands 213. Each of the one or more reinforcement bands 213 can be a monolithic band or can be an adjustable band, for example, adjustable by a user. For example, an overlapped band that can be tightened or loosened. In some embodiments, the bands can be configured to automatically adjust, for example in response to the stresses on the outer wall 11.

The total mass of the portable automated infusion system 10, 110, 210 can be between 45 and 950 grams, or between 75 grams and 750 grams. The microcontroller 7, 104, 204 and the memory 39 can be configured to operate with an artificial intelligence (AI) system, to continually update and improve operation.

In some embodiments, the portable automated infusion system 10, 110, 210 can further include a downstream warming device (not shown) for warming blood to a desired temperature for transfusion. In some embodiments, the portable automated infusion system 10, 110, 210 can further comprise a temperature sensor, such as a thermocouple, thermistor, or RTD, to measure the temperature of the blood, or any other element or component. The portable automated infusion system 10, 110, 210 not only allows for safe and repeatable transfusions in the most difficult circumstances and locations, but is also relatively lightweight, reusable, rechargeable, and does not comprise many different components which can be misplaced. In some embodiments, all of the elements of the portable automated infusion system 10, 110, 210 are together in one carriable piece. Within a few minutes, a patient can be set up, transfusion can begin, the portable automated infusion system 10, 110, 210 will do the rest of the work of transfusion automatically, without the aid of any user.

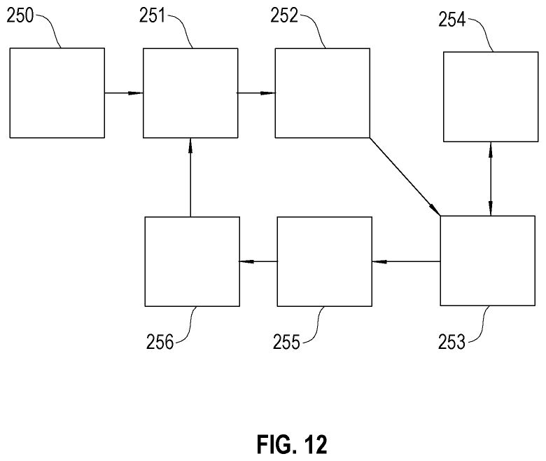

FIG. 12 illustrates a feedback loop for the portable automated infusion system 10, 110, 210. Upon initiation of the system 10, 110, 210 (e.g., a user pushing a “start” or “on” button 250), the user can then adjust a setpoint 251, for example, utilizing toggle buttons 4, 108, 208. The setpoint 251 is stored in the memory 39. The setpoint 251 can comprise a target fluid pressure. Alternatively, the setpoint 251 can comprise a pump motor speed, an input voltage supplied to pump, a fluid flow rate, a fluid flow rate rise time, and a fluid pressure rise time. The setpoint 251 is achieved via operation of a motor and/or air pump 252, utilizing external air (or other fluid) 254, to achieve an actual pressure 253 within the annulus 22. This pressure 253 is measured in real time by a pressure sensor 255 which outputs data. The controller 256 receives the data from the sensor 255, and compares the measured value (e.g., of actual pressure 253) with the setpoint 251. The differential of the actual pressure 253 and the setpoint 251 pressure is utilized by the controller 256 to determine whether to add more air within the annulus 22, or allow a controlled amount of air to leave the annulus 22.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

For purposes of the present disclosure and appended claims, the conjunction “or” is to be construed inclusively (e.g., “an apple or an orange” would be interpreted as “an apple, or an orange, or both”; e.g., “an apple, an orange, or an avocado” would be interpreted as “an apple, or an orange, or an avocado, or any two, or all three”), unless: (i) it is explicitly stated otherwise, e.g., by use of “either . . . or,” “only one of,” or similar language; or (ii) two or more of the listed alternatives are mutually exclusive within the particular context, in which case “or” would encompass only those combinations involving non-mutually-exclusive alternatives. For purposes of the present disclosure and appended claims, the words “comprising,” “including,” “having,” and variants thereof, wherever they appear, shall be construed as open-ended terminology, with the same meaning as if the phrase “at least” were appended after each instance thereof.

1-103. (canceled)

104. A portable automated infusion system comprising:
a sleeve comprising a first end, a second end, a longitudinal axis, and an internal opening extending along the longitudinal axis from the first end, the internal opening configured to allow a blood bag to be close-fittingly carried therein, the sleeve comprising:
an outer wall having substantially low overall distensibility, the outer wall extending 360° around the longitudinal axis and configured to surround the blood bag, when the blood bag is placed within the internal opening of the sleeve;
an inner wall having a first end and a second end, the first end of the inner wall in a sealing arrangement with a first portion of the outer wall at or adjacent the first end sleeve and the second end of the inner wall in a sealing arrangement with a second portion of the outer wall spaced from the first portion in a direction toward the second end of the sleeve, the inner wall having an interior portion defining the internal opening; and
a substantially annular space between the outer wall and the inner wall;
a pump coupled to the sleeve and configured to pump a fluid into the annular space;
a pressure sensor configured to measure fluid pressure within the annular space and to output a signal related to the measured fluid pressure; and
a controller configured to receive the output signal and configured to control operation of the pump.

105-109. (canceled)

110. The system of claim 104, wherein the sleeve is reusable.

111. The system of claim 104, wherein the first end of the sleeve is configured to be an upper end and wherein the second end of the sleeve is configured to be a lower end.

112. The system of claim 104, wherein the internal opening extends completely through the sleeve between the first end and the second end.

113. The system of claim 104, wherein the outer wall in a transverse cross-section to the longitudinal axis has a generally elliptical shape.

114. The system of claim 104, wherein the outer wall in a transverse cross-section to the longitudinal axis has a generally oval shape.

115. The system of claim 104, wherein the outer wall in a transverse cross-section to the longitudinal axis has a generally rectangular shape.

116. The system of claim 104, wherein the outer wall comprises a front portion and a back portion, the back portion statically coupled to the back portion.

117. The system of claim 116, wherein the front portion is connected to the back portion with a fastener.

118. The system of claim 104, wherein the front portion is bonded to the back portion.

119. The system of claim 104, further comprising a band closely extending around an exterior of the outer wall around the longitudinal axis, the band configured to provide added resistance to distension of the outer wall.

120. The system of claim 104, wherein the inner wall comprises a polymer selected from the list consisting of: low-density polyethylene (LDPE), medium-density polyethylene (MDPE), a polyolefin, a polyamide, a polyether block amide, polyvinyl chloride, and polyurethane.

121. The system of claim 104, further comprising:
a one-way valve configured to allow external air to be pumped into the annular space by the pump while not allowing the air pumped into the annular space to substantially exit the annular space.

122. The system of claim 104, further comprising:
a memory configured to store one or more routines for pump operation, wherein the one or more routines comprise a pattern of pump speed over time.

123. The system of claim 104, further comprising:
a memory configured to store one or more routines for pump operation, wherein the one or more routines comprise a pattern for target fluid pressure of the annular space.

124. The system of claim 104, further comprising:
a memory configured to store one or more routines for pump operation, wherein the one or more routines comprise a target fluid flow rate.

125. The system of claim 104, further comprising:
a memory configured to store one or more routines for pump operation, wherein the one or more routines comprise a target flow velocity.

126. The system of claim 125, further comprising:
a Doppler flow sensor configured to measure flow velocity of fluid entering the annular space.

127. The system of claim 104, further comprising:
a valve coupled between ambient environment and the annular space, wherein the controller is configured to open and close the valve, to allow fluid to exit the external space.

128. The system of claim 127, wherein the controller is configured to cause the valve to open and close, to allow fluid to exit the external space.
